(19) United States
(12) Patent Application Publication
(10) Pub. No.: US 2009/0325229 A1
(43) Pub. Date: Dec. 31, 2009

(54) Process for Globular Adiponectin Production

(76) Inventors: Pierre Graber, Saint-Cergue (CH); Andrew Topping, MIlford, MA (US); Philippe Baumgartner, Cleveland (GB); Ewan Robert Robertson, Cleveland (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/304,206

(22) PCT Filed: Jun. 19, 2007
(86) PCT No.: PCT/EP2007/005391

§ 371 Date: Apr. 28, 2009

Related U.S. Application Data
(60) Provisional application No. 60/816,446, filed on Jun. 26, 2006.

(30) Foreign Application Priority Data
Jun. 20, 2006 (EP) 06115719.4
Jul. 6, 2006 (EP) 06116705.2

(51) Int. Cl. C12P 21/00

(52) U.S. Cl. 435/69.1

(57) ABSTRACT

This invention relates to methods for use in industrial production of recombinant globular Adiponectin (gAdiponectin). Specifically, the present invention provides a purification process suitable for production of high amounts of pure gAdiponectin. gAdiponectin is expressed in E. coli, and a purification method with which about 30 grams of gAdiponectin could be obtained from 100 liters of cell culture has been set up.

### FIELD OF THE INVENTION

This invention relates to methods for use in industrial production of recombinant globular Adiponectin (gAdiponectin). Specifically, the present invention provides a purification process suitable for production of high amounts of pure gAdiponectin. gAdiponectin is expressed in E. coli, and a purification method with which about 30 grams of gAdiponectin could be obtained from 100 liters of cell culture has been set up.

### BACKGROUND OF THE INVENTION

### 1. Adiponectin as a Pharmaceutical

Adipose tissue, while long known for its capacity to store fat, has an important role as the source for a number of hormones and paracrine mediators, including resistin, adipsin, leptin, and TNF-α. Collectively, these molecules are termed adipokines, to emphasize their role as hormone and site of synthesis. Adiponectin, also referred to as Acrp30 or ApM-1, is one such adipokine and is produced by adipose tissue. However, Adiponectin cannot be considered as a typical hormone because its concentration in plasma is not within the hormonal range. Indeed, Adiponectin concentrations in plasma vary from 2 to 18 μg/ml, whereas hormone concentrations are typically below or within the ng/ml range.

Mouse Adiponectin was first identified in 1995 (Scherer et al., 1995), and was shown to be up-regulated over 100-fold during adipocyte differentiation. The human homologue was identified in 1996 (Maeda et al., 1996). Adiponectin contains an amino-terminal signal sequence, followed by a central region comprising collagen repeats, and a carboxyl-terminal domain with homology to the globular complement factor C1q. This latter domain is commonly referred to as the “globular head” of Adiponectin (gAdiponectin). Several studies have specifically focused on fragments of Acpr30 comprising the globular head (PCT publication No. WO 01/51645) (Fruebis et al., 2001). gAdiponectin polypeptides associate as trimers and are pharmaceutically active (Fruebis et al., 2001; Pajvani et al., 2003)

Studies have demonstrated that Adiponectin is linked to obesity and type II diabetes. Genetic data have demonstrated linkage of type II diabetes with non-coding Single Nucleotide Polymorphisms (SNPs) located within the Adiponectin gene in a Japanese cohort of patients (Hara et al., 2002). It was further demonstrated that missense mutations affecting the globular head are correlated with serum levels of Adiponectin (Kondo et al., 2002).

In addition, serum levels of Adiponectin are decreased in several models of obesity, including leptin-deficient mice, leptin-receptor deficient mice, and monkey models (Hu et al., 1996; Yamauchi et al., 2001). In human studies, Adiponectin levels are inversely correlated to both diabetes and obesity, and they are further reduced in patients with coronary artery disorder (Arita et al., 1999). Further evidence for a causal relationship between reduced levels of Adiponectin and development of insulin resistance and type II diabetes was obtained by Lindsay et al., who showed that individuals in the Pima Indian population who had lower serum levels of Adiponectin were more likely to develop type II diabetes than those with higher levels (Lindsay et al., 2002). In 2002, it was found that homozygous Adiponectin-deficient mice were not hyperglycemic when maintained on a normal diet, but they exhibited reduced clearance of serum free fatty acid. When switched to a high-fat, high-sucrose diet, they exhibited severe insulin resistance and demonstrated increased weight gain relative to control animals (Maeda et al., 2002).

In addition to its pivotal role in obesity and diabetes, Adiponectin has been suggested to play a role in other disorders. Specifically, association of serum or plasma levels of Adiponectin with polycystic ovary syndrome (Panidis et al., 2003), endometrial cancer (Petridou et al., 2003), preeclampsia (Ramsay et al., 2003) and the nephritic syndrome (Zoccali et al., 2003) has been observed. Adiponectin has also been shown to display anti-inflammatory properties (Yokota et al., 2000) and to alleviate fatty liver diseases in mice (Xu et al., 2003).

In addition, EP application No. 05 107 038.1 teaches that gAdiponectin exhibits anti-coagulant and/or anti-aggregant properties. Therefore, gAdiponectin is useful for the treatment and/or prevention of venous and arterial thrombosis, tumor implantation, tumor seeding, metastasis and hypertensive disorders of the pregnancy.

### 2. Purification of Proteins from Inclusion Bodies (IBs)

The purification process of recombinant proteins expressed in E. coli as insoluble inclusion bodies usually comprises the following steps:
Solubilization of the IBs in a solution comprising guanidine;
Refolding of the protein by dilution in a solution comprising urea;
Concentration of the protein; and
Filtration to remove misfolded and aggregated proteins.

Nevertheless, the conditions and additional steps are specific for each protein and need to be developed on a case-by-case basis.

### 3. Production of Adiponectin and of Globular Adiponectin

It is well known in the art that there are technical difficulties in producing active Adiponectin polypeptides. This is for example illustrated by the abstract of a presentation given by Dr. Violand at IBC's conference “Engineering Proteins and Antibodies for Advanced Biotherapeutics” that was held in Basel in 2005, where it is stated that Dr. Violand's group were unsuccessful in using E. coli as an expression system for producing Adiponectin polypeptides.

Adiponectin polypeptides have been produced in small quantities from E. coli (see e.g., Arita et al., 1999), human cell lines (see e.g., Berg et al., 2001) and insect cell lines (see e.g., Neumeier et al., 2006). However, all publications mentioning Adiponectin polypeptide production relate to processes allowing to obtain only small amounts of polypeptides. In addition, most of these publications disclose the purification of a fusion protein comprised of a Adiponectin polypeptide and of a tag such as a His-Tag, wherein the tag allows purification of the fusion protein (see e.g. Fruebis et al., 2001; Liu et al., 2006).

More specifically, Liu et al. (2006) discloses a method for purifying gAdiponectin polypeptides from inclusion bodies. However, this gAdiponectin polypeptide comprises a 6-His-Tag at its N-terminal extremity and is purified using a Ni⁺-affinity anti-His-Tag chromatography. The protein to be purified must therefore comprise a Tag and cannot correspond to a fragment of a naturally-occurring Adiponectin. In addition, the purity is only of 90% and the article is totally silent on the yield of the disclosed purification process.

There is thus a need for a purification process of gAdiponectin polypeptides, wherein the process allows obtaining high yields of active and pure gAdiponectin polypeptides.

### SUMMARY OF THE INVENTION

The present invention provides methods of producing gAdiponectin polypeptides from E. coli that allows obtaining high yields of active and pure gAdiponectin polypeptides.

Accordingly, the present invention is directed to method of producing a recombinant polypeptide wherein said method comprises the steps of:
a) Cultivation of recombinant E. coli cells expressing said recombinant polypeptide;
b) Lysis of said cells;
c) Recovery of inclusion bodies (IBs) comprising said recombinant polypeptides;
d) Washing of said IBs in a first solution;
e) Solubilization of said IBs in a second solution;
f) Buffer exchange of the solubilized IBs into a third solution;
g) Refolding of said recombinant polypeptide by adding the solution obtained at the end of step (f) into a fourth solution;
h) Concentration of said recombinant polypeptides by passing the solution obtained at the end of step (g) through an anion exchange chromatography column;
i) Recovery of the fractions comprising said recombinant polypeptides; and, optionally
j) Passage of the fractions obtained at the end of step (i) through a size exclusion chromatography column; and,
k) Recovery of the fractions comprising said recombinant polypeptides.

wherein said process is characterized in that:
(i) said recombinant polypeptide is a polypeptide comprising the globular head of Adiponectin (gAdiponectin);
(ii) said second solution comprises guanidine and its pH is acidic;
(iii) said third solution comprises urea and its pH is acidic;
(iv) said solution obtained at the end of step (f) is added progressively into said fourth solution; and
(v) the pH of said fourth solution is basic.

### BRIEF DESCRIPTION OF THE FIGURES

FIG. 1 shows the rshgAd polypeptides obtained by the process of Example 2 (FIG. 1 A) and of Example 3 (FIG. 1 B).

FIG. 2 is a scheme comparing the processes of Examples 2 and 3.

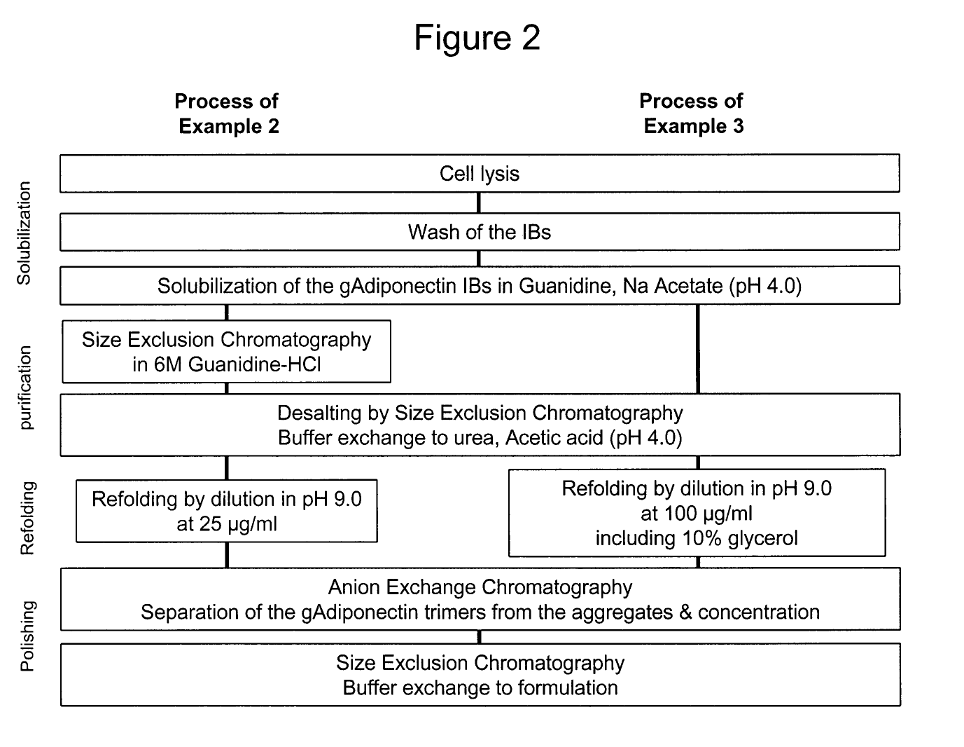

FIG. 3 shows the effect of the pH of the solution used for solubilizing the inclusion bodies (“second solution”) on the quality of the rshgAd proteins obtained by the process of Example 2.

FIG. 4 shows the effect of the solubilization agent of the solution used for solubilizing the inclusion bodies (“second solution”) on the quality of the rshgAd proteins obtained by the process of Example 2.

FIG. 5 shows the effect of the pH of the solution used during the refolding step (“fourth solution”) on the quality of the rshgAd proteins obtained by the process of Example 2.

### DETAILED DESCRIPTION OF THE INVENTION

The present invention stems from the finding of a method of producing and/or purifying recombinant gAdiponectin polypeptides in such a way as to obtain pure gAdiponectin polypeptides that may be administered to humans as a pharmaceutical. Example 2 and 3 teach two different embodiments of the method of the present invention.

A first aspect relates to a method of producing a recombinant polypeptide wherein said method comprises the steps of:
a) Cultivation of recombinant E. coli cells expressing said recombinant polypeptide;
b) Lysis of said cells;
c) Recovery of inclusion bodies (IBs) comprising said recombinant polypeptides;
d) Washing of said IBs in a first solution;
e) Solubilization of said IBs in a second solution;
f) Buffer exchange of the solubilized IBs into a third solution;
g) Refolding of said recombinant polypeptide by adding the solution obtained at the end of step (f) into a fourth solution;
h) Concentration of said recombinant polypeptides by passing the solution obtained at the end of step (g) through an anion exchange chromatography column;
i) Recovery of the fractions comprising said recombinant polypeptides; and, optionally
j) Passage of the fractions obtained at the end of step (i) through a size exclusion chromatography column; and,
k) Recovery of the fractions comprising said recombinant polypeptides.
   wherein said process is characterized in that:
(i) said recombinant polypeptide is a polypeptide comprising the globular head of Adiponectin (gAdiponectin);
(ii) said second solution comprises guanidine and its pH is acidic;
(iii) said third solution comprises urea and its pH is acidic;
(iv) said solution obtained at the end of step (f) is added progressively into said fourth solution; and
(v) the pH of said fourth solution is basic.

Such a method is further referred to as “method according to the invention”.

The buffer exchange of the solubilized IBs from the second into the third solution (i.e., step (f) of the method according to the invention) may be carried out using any method well-known in the art such as, e.g., ultrafiltration, dialysis or using a column that is suitable for desalting.

In a preferred embodiment, the buffer exchange of the solubilized IBs into a third solution comprises the steps of:
(i) Passage of the solubilized IBs through a size exclusion chromatography column equilibrated with said third solution;
(ii) Recovery of the fractions comprising said gAdiponectin polypeptides; and, optionally
(iii) Pooling of said fractions obtained at step (ii).
   Step (iii) of this embodiment is not carried out when only one fraction comprise gAdiponectin polypeptides.

The method according to the invention may further comprise the step of filtrating the solution obtained at the end of step (g).

The method according to the invention may further comprise the step of concentrating and/or filtrating the solution obtained at the end of step (i).

The method according to the invention may further comprise the step of concentrating and/or filtrating the solution obtained at the end of step (k).

The method according to the invention may further comprise the step of formulating said gAdiponectin polypeptide into a pharmaceutical composition.

In a preferred embodiment, the purity of the recombinant gAdiponectin polypeptides in the fractions obtained at the end of step (k) is of at least 90%, 91%, 92%, 93%, 94%, 95%, 96%, 97%, 98, 99% or 100%. Preferably, said purity is of at least 95%.

As used herein, the phrase “added progressively” means that two solutions are not melted together in one single step but to the contrary, that a solution is added to another solution either step by step or continuously over a period of time. Preferably, said period of time is of about 24, 20, 18, 16, 14, 12, 10, 8, 6 or 4 hours. Most preferably, said period of time is of about 12 hours. This addition of a solution to another is preferably carried out using a pump. Infinite dilution is the simplest technique that can be applied to add progressively a solution to another at the industrial level, and consists in the addition of droplets of a solution to a bath of another solution. Infinite dilution of the third solution to the fourth solution is preferably carried out overnight.

As used herein, the term “inclusion body” or “IB” refers to insoluble aggregates of denatured, unfolded and/or misfolded protein.

As used herein, the phrase “refolding of a polypeptide” refers to a process by which a protein which has been denatured, unfolded and/or misfolded is forced to adopt its native functional structure. The basic principle of protein refolding is the removal of denaturant from the system. Proteins are refolded by an exchange of buffers—from denaturant-containing buffer (solubilization buffer) to no denaturant-containing buffer (refolding buffer).

1. Adiponectin Polypeptides

The term “Adiponectin polypeptide”, as used herein, refers to a full-length or mature Adiponectin protein and to fragments thereof having biological activity. The term also encompasses muteins of SEQ ID NO: 1. The term further encompasses homologues of a human Adiponectin polypeptide in other species. However, a human or a mouse Adiponectin is preferably used in the methods and uses of the present invention. The Adiponectin polypeptide may correspond to a fused protein, a functional derivative, an active fraction or fragment, a circularly permutated derivative or a salt of a polypeptide comprising SEQ ID NO: 1, or a mutein thereof. Preferably, Adiponectin has biological activity.

As used herein, the term “biological activity” of an Adiponectin polypeptide refers to an activity selected from the group of an anti-diabetic, anti-obesity, anti-thrombotic, anti-coagulant and anti-aggregant activity. Other activities include stimulation of muscle lipid and/or stimulation of free fatty acid oxidation. The biological activity of an Adiponectin polypeptide can be assessed as described, e.g., in WO 01/51645 or in EP patent application No. 05 107 038.1.

In the methods of the present invention, the Adiponectin polypeptide comprises the globular head of Adiponectin. The term “gAdiponectin” is synonymous with the term “globular head of Adiponectin”. As used herein, these terms refer to a polypeptide comprising a fragment of Adiponectin, said fragment (i) comprising amino acids 115 to 244 of SEQ ID NO: 1 and (ii) lacking amino acids 1 to 70 of SEQ ID NO: 1. The term also encompasses muteins of such gAdiponectin polypeptides. The term further encompasses homologues of a human gAdiponectin polypeptide in other species. However, a human gAdiponectin is preferably used in the methods and uses of the present invention. The gAdiponectin polypeptide may correspond to a fused protein, a functional derivative, an active fraction or fragment, a circularly permutated derivative or a salt of a polypeptide comprising amino acids 115 to 244 of SEQ ID NO: 1 and lacking amino acids 1 to 70 of SEQ ID NO: 1, or a mutein thereof. Preferably, gAdiponectin has biological activity.

In most preferred embodiments, the gAdiponectin polypeptide consists of amino acids 1 to 138 of SEQ ID NO: 2, or of amino acids 2 to 138 of SEQ ID NO: 2.

In a preferred embodiment of the present invention, the gAdiponectin polypeptide is selected from the group consisting of:
a) A polypeptide comprising amino acids 115 to 244 of SEQ ID NO: 1;
b) A polypeptide comprising amino acids 2 to 138 of SEQ ID NO: 2;
c) A polypeptide comprising SEQ ID NO: 2;
d) A polypeptide comprising amino acids 106 to 244 of SEQ ID NO: 1;
e) A polypeptide comprising amino acids 79 to 244 of SEQ ID NO: 1;
f) A mutein of any of (a) to (e), wherein the amino acid sequence has at least 75%, 80%, 85%, 90%, 95%, 96%, 97%, 98% or 99% identity to at least one of the sequences in (a) to (e);
g) A mutein of any of (a) to (e) which is encoded by a DNA sequence which hybridizes to the complement of the native DNA sequence encoding any of (a) to (e) under moderately stringent conditions or under highly stringent conditions;
h) A mutein of any of (a) to (e) wherein any changes in the amino acid sequence are conservative amino acid substitutions to the amino acid sequences in (a) to (e);
i) A salt or a fused protein, functional derivative, active fraction or circularly permutated derivative of any of (a) to (h).
   wherein said gAdiponectin polypeptide does not comprise amino acids 1 to 70 of SEQ ID NO: 1.

In one embodiment, the gAdiponectin polypeptide in accordance with the present invention is selected from the gAdiponectin polypeptides disclosed in PCT publication No. WO 01/51645.

In a preferred embodiment of the present invention, the gAdiponectin polypeptide in accordance with the present invention comprises a contiguous span of SEQ ID NO: 1 starting at amino acid position 105, 106, 107, 108, 109, 110, 111, 112, 113, 114 or 115 and ending at amino acid position 244 of SEQ ID NO: 1. Most preferably, the gAdiponectin polypeptide in accordance with the present invention comprises a contiguous span of SEQ ID NO: 1 starting at amino acid position 107, 108, 109, 110 or 111 and ending at amino acid position 244 of SEQ ID NO: 1.

Alternatively, the gAdiponectin in accordance with the present invention comprises a contiguous span of SEQ ID NO: 1 starting at amino acid position 75, 76, 77, 78, 79, 80, 81, 82, 83, 84, 85, 86, 87, 88, 89, 90, 91 or 92 and ending at amino acid position 244 of SEQ ID NO: 1. Most preferably, the gAdiponectin polypeptide in accordance with the present invention comprises a contiguous span of SEQ ID NO: 1 starting at amino acid position 78, 79 or 80 and ending at amino acid position 244 of SEQ ID NO: 1.

A gAdiponectin polypeptide in accordance with the invention does not comprise amino acids 1 to 70 of SEQ ID NO: 1. Preferably, it does not comprise amino acids 1 to 75, 1 to 80, 1 to 90, 1 to 95, 1 to 100, 1 to 105, 1 to 110 or 1 to 113 of SEQ ID NO: 1.

The person skilled in the art will further appreciate that splice variants, allelic variants, muteins, fragments, salts, homologues in other species, fused proteins, functional derivatives, active fractions and circularly permutated derivatives of the gAdiponectin polypeptides of SEQ ID NO: 2 will retain a similar, or even better, biological activity than gAdiponectin polypeptides of SEQ ID NO: 2.

Preferred active fractions have an activity which is equal or better than the activity of gAdiponectin polypeptides of SEQ ID NO: 2, or which have further advantages, such as a better stability or a lower toxicity or immunogenicity, or they are easier to produce in large quantities, or easier to purify. The person skilled in the art will appreciate that muteins, active fragments and functional derivatives can be generated by cloning the corresponding cDNA in appropriate plasmids and testing them in the co-culturing assay, as mentioned above.

The gAdiponectin polypeptides according to the present invention are produced recombinantly. Recombinant expression is carried out in prokaryotic expression systems such as, e.g., E. coli or B. subtilis, or in lower eukaryotes such as, e.g., yeast or Aspergillus.

As used herein the term “muteins” refers to analogs of a gAdiponectin polypeptide comprising amino acids 115 to 244 of SEQ ID NO: 1 and lacking amino acids 1 to 70 of SEQ ID NO: 1 in which one or more of the amino acid residues of said polypeptide are replaced by different amino acid residues, or are deleted, or one or more amino acid residues are added to the natural sequence of said polypeptide, without changing considerably the activity of the resulting products as compared with the polypeptide of SEQ ID NO: 2. These muteins are prepared by known synthesis and/or by site-directed mutagenesis techniques, or any other known technique suitable therefore. The term “muteins” encompasses naturally occurring allelic variants and naturally occurring splice variants or cleavage products of an Adiponectin polypeptide of SEQ ID NO: 1.

Muteins of a gAdiponectin polypeptide comprising amino acids 115 to 244 of SEQ ID NO: 1 and lacking amino acids 1 to 70 of SEQ ID NO: 1, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Muteins in accordance with the present invention include proteins encoded by a nucleic acid, such as DNA or RNA, which hybridizes to DNA or RNA, which encodes a gAdiponectin polypeptide comprising amino acids 115 to 244 of SEQ ID NO: 1 and lacking amino acids 1 to 70 of SEQ ID NO: 1, under moderately or highly stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. ° F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

In a preferred embodiment, any such mutein has at least 40% identity with the sequence of a gAdiponectin polypeptide comprising amino acids 115 to 244 of SEQ ID NO: 1 and lacking amino acids 1 to 70 of SEQ ID NO: 1. More preferably, it has at least 50%, 55%, 60%, 65%, 70%, 75%, 80%, 85% or, most preferably, at least 90%, 95%, 96%, 97%, 98% or 99% identity thereto.

In another preferred embodiment, such mutein has at least 40% identity with the sequence of a gAdiponectin polypeptide of SEQ ID NO: 2. More preferably, it has at least 50%, 55%, 60%, 65%, 70%, 75%, 80%, 85% or, most preferably, at least 90%, 95%, 96%, 97%, 98% or 99% identity thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called “global alignment”), that is particularly suitable for sequences of the same or very similar length, or over shorter, defined lengths (so-called “local alignment”), that is more suitable for sequences of unequal length. In the frame of the present invention, the “% of identity” refers to the global percent of identity that has been determined over the whole length of each of the sequences being compared.

Known computer programs may be used to determine whether any particular polypeptide is a percentage identical to a sequence of the present invention. Such algorithms and programs include, e.g. TBLASTN, BLASTP, FASTA, TFASTA, and CLUSTALW (Altschul et al., 1990; Altschul et al., 1997; Higgins et al., 1996; Pearson and Lipman, 1988; Thompson et al., 1994). Protein and nucleic acid sequence homologies are preferably evaluated using the Basic Local Alignment Search Tool (“BLAST”), which is well known in the art (Altschul et al., 1990; Altschul et al., 1997; Karlin and Altschul, 1990).

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. The scoring matrix used may be the BLOSUM62 matrix (Gonnet et al., 1992; Henikoff and Henikoff, 1993). The PAM or PAM250 matrices may also be used (See, e.g., Schwartz and Dayhoff, eds, (1978) Matrices for Detecting Distance Relationships: Atlas of Protein Sequence and Structure, Washington: National Biomedical Research Foundation). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (Karlin and Altschul, 1990). The BLAST programs may be used with the default parameters or with modified parameters provided by the user.

A preferred method for determining the best overall match between a query sequence (a sequence of the present invention) and a subject sequence, also referred to as a global sequence alignment, can be determined using the FASTDB computer program based on the algorithm of Brutlag (Brutlag et al., 1990). In a sequence alignment the query and subject sequences are both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group=25 Length=0, Cutoff Score=1, Window Size=sequence length, Gap Penalty=5, Gap Size Penalty=0.05, Window Size=247 or the length of the subject amino acid sequence, whichever is shorter.

If the subject sequence is shorter than the query sequence due to N- or C-terminal deletions, not because of internal deletions, the results, in percent identity, must be manually corrected because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, that are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query amino acid residues outside the farthest N- and C-terminal residues of the subject sequence.

For example, a 90 amino acid residue subject sequence is aligned with a 100-residue query sequence to determine percent identity. The deletion occurs at the N-terminus of the subject sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%.

Preferred changes for muteins in accordance with the present invention are what are known as “conservative” substitutions. Conservative amino acid substitutions of gAdiponectin polypeptides in accordance with the present invention may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Trp |

Examples of production of amino acid substitutions in polypeptides which can be used for obtaining muteins of a gAdiponectin polypeptide of SEQ ID NO: 2 include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al).

The term “fused protein” refers to a polypeptide comprising amino acids 115 to 244 of SEQ ID NO: 1 and lacking amino acids 1 to 70 of SEQ ID NO: 1 or a mutein thereof fused with another protein, which e.g. has an extended residence time in body fluids. The gAdiponectin moiety may be fused to another protein, polypeptide or the like, e.g. an immunoglobulin or a fragment thereof. Immunoglobulin Fc portions are particularly suitable for production of di- or multi-meric Ig fusion proteins. The gAdiponectin moiety in accordance with the present invention may e.g. be linked to portions of an immunoglobulin in such a way as to produce a gAdiponectin polypeptide dimerized by the Ig Fc portion. Alternatively, the sequence of the gAdiponectin moiety is fused to a signal peptide and/or to a leader sequence allowing enhanced secretion. The leader sequence may for example corresponds to the IgSP-tPA pre-propeptide disclosed in PCT publication WO 2005/030963.

In one embodiment, the gAdiponectin polypeptide in accordance with the present invention consists of a fragment of full-length Adiponectin. Alternatively, the gAdiponectin polypeptide in accordance with the present invention is a fused protein comprising a carrier molecule, a peptide or a protein that promotes the crossing of the blood brain barrier, and/or comprising a carrier molecule, a peptide, a Tag such as a His-tag or a protein that increases half-life. Alternatively, the gAdiponectin polypeptide does not comprise any Tag.

The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the gAdiponectin sequence and the protein to which it is fused. The resulting fusion protein has improved properties, such as an extended residence time in body fluids (half-life), or an increased specific activity, increased expression level. The Ig fusion may also residence time in body fluids.

“Functional derivatives” as used herein, cover derivatives of a polypeptide comprising amino acids 115 to 244 of SEQ ID NO: 1 and lacking amino acids 1 to 70 of SEQ ID NO: 1 or a mutein thereof, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of a polypeptide of SEQ ID NO: 2, and do not confer toxic properties on compositions containing it.

These derivatives may, for example, include polyethylene glycol side-chains, which may mask antigenic sites and extend the residence of a naturally occurring gAdiponectin polypeptide in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of a polypeptide comprising amino acids 115 to 244 of SEQ ID NO: 1 and lacking amino acids 1 to 70 of SEQ ID NO: 1 or a mutein thereof, the present invention covers any fragment or precursors of the polypeptide chain of the protein molecule alone or together with associated molecules or residues linked thereto, e.g. sugar or phosphate residues, or aggregates of the protein molecule or the sugar residues by themselves, provided said fraction has substantially similar activity to a gAdiponectin polypeptide of SEQ ID NO: 2.

The term “salts” herein refers to both salts of carboxyl groups and to acid addition salts of amino groups of a polypeptide comprising amino acids 115 to 244 of SEQ ID NO: 1 and lacking amino acids 1 to 70 of SEQ ID NO: 1 or a mutein thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of a gAdiponectin polypeptide of SEQ ID NO: 2.

Functional derivatives may be conjugated to polymers in order to improve the properties of the protein, such as the stability, half-life, bioavailability, tolerance by the human body, or immunogenicity. To achieve this goal, the gAdiponectin polypeptide may be linked e.g. to Polyethlyenglycol (PEG). PEGylation may be carried out by known methods, described in WO 92/13095, for example.

Therefore, in a preferred embodiment of the present invention, the gAdiponectin polypeptide in accordance with the present invention is PEGylated.

Adiponectin exists as different species of different apparent molecular weight (Scherer et al., 1995). The structure of these species was investigated by Tsao et al. (2002, 2003). Adiponectin polypeptides exist as monomers, trimers, hexamers and HMW species. “HMW species of Adiponectin” refers to a complex of Adiponectin polypeptides comprising more than six Adiponectin polypeptides. The apparent molecular mass of murine HMW species of Adiponectin is of about 630 kDa.

The method of the present invention is preferably used to produce trimers of gAdiponectin polypeptides.

In a preferred embodiment, the gAdiponectin polypeptides are formulated into a pharmaceutical composition at the end of the production process according to the present invention.

Such pharmaceutical compositions may be useful for prevention and/or treatment of a disease such as, e.g., obesity, type II diabetes, insulin resistance, hypercholesterolemia, hyperlipidemia, dyslipidemia, syndrome X, atherosclerosis, thromboembolism, deep vein thrombosis (DVT), thrombophlebitis, venous claudication, venous thromboembolism or venous thromboembolism (VTE), pulmonary thromboembolism (PTE), pulmonary embolism (PE), venous thrombosis, deep vein thrombus, deep venous thrombus, obstructed venous outflow, chronic venous insufficiency (CVI), postphlebitic syndrome, coronary arterial thrombosis, unstable angina, stable angina or myocardial infarction, ischemic stroke, intermittent claudication, atrial fibrillation, ischemic events, acute and chronic heart failure, hypertensive disorders of the pregnancy, gestational hypertension (GH), nonproteinuric gestational hypertension, preeclampsia, nonproteinuric preeclampsia, eclampsia, nonproteinuric eclampsia, pregnancy-induced hypertension (PIH), polycystic ovary syndrome, nephritic syndrome, inflammatory diseases, tumor implantation, tumor seeding, tumor metastasis. As used herein, the term “tumor” encompasses, e.g., colon cancer, endometrial cancer, breast cancer, melanomas, myelomas, sarcomas, lymphomas, leukemias such as chronic or acute lymphocytic leukemia, carcinomas such as non-small cell lung carcinoma and breast carcinoma.

Such pharmaceutical compositions comprise (i) a therapeutically effective amount of an gAdiponectin polypeptide in accordance with the invention, and (ii) a pharmaceutically acceptable carrier. The definition of “pharmaceutically acceptable carrier” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, intrathecal, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

The therapeutically effective amounts of the active protein(s) will be a function of many variables, including the type of protein, the affinity of the protein, any residual cytotoxic activity exhibited by the antagonists, the route of administration, the clinical condition of the patient (including the desirability of maintaining a non-toxic level of endogenous gAdiponectin activity).

A “therapeutically effective amount” is such that when administered, the gAdiponectin polypeptide in accordance with the present invention exerts a beneficial effect on at least one of the diseases listed hereabove. The dosage administered, as single or multiple doses, to an individual will vary depending upon a variety of factors, including gAdiponectin pharmacokinetic properties, the route of administration, patient conditions and characteristics (sex, age, body weight, health, size), extent of symptoms, concurrent treatments, frequency of treatment and the effect desired.

The gAdiponectin polypeptide in accordance with the invention can preferably be used in an amount of about 0.01 to 10 mg/kg or about 0.05 to 5 mg/kg or body weight or about 0.1 to 3 mg/kg of body weight or about 1 to 2 mg/kg of body weight. Further preferred amounts of gAdiponectin polypeptides are amounts of about 0.01 to 1000 μg/kg of body weight or about 0.1 to 100 μg/kg. Further preferred amounts of gAdiponectin polypeptides are amounts amounts of about 1 to 10 μg/kg of body weight or about 10 to 50 μg/kg of body weight.

According to the invention, the gAdiponectin polypeptide in accordance with the invention can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

2. Process of the First Embodiment

This embodiment is illustrated by Example 3.

### 2.1. Wash of the IBs

A preferred embodiment is directed to a method of producing a recombinant gAdiponectin polypeptide according to the invention, wherein said first solution comprises ethanol. Ethanol is very efficient for washing IBs comprising gAdiponectin polypeptides and allows reducing the number of washing steps. Preferably, said first solution comprises about 30%, 25%, 20%, 15%, 10% or 5% ethanol. More preferably, said first solution comprises about 20% ethanol. Most preferably, said first solution is a 100 mM Tris/HCl solution at pH 7.5.

### 2.2. Solubilization of the IBs

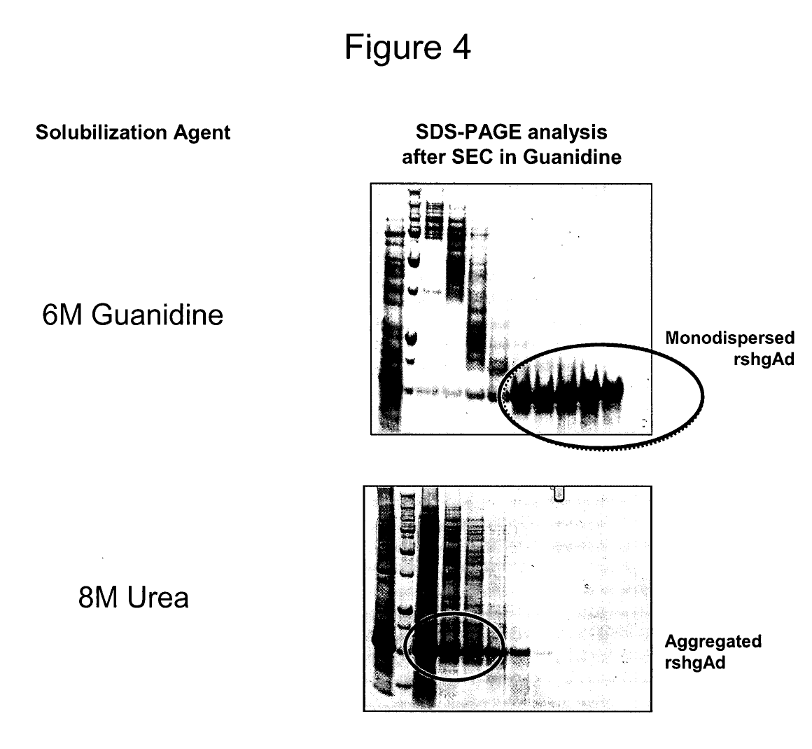

A preferred embodiment is directed to a method according to the invention, wherein said second solution comprises about 6 M, 5 M or 4 M Guanidine-HCl. Preferably, said second solution comprises about 6 M Guanidine-HCl. Analysis of the preparative size exclusion chromatography (SEC) run under denaturing conditions by SDS-PAGE showed that the solubilization of the inclusion bodies was efficient with Guanidine-HCl, where most of the material is recovered as monodisperse molecules (see FIG. 4). On the other hand, solubilization of the inclusion bodies in urea was not as efficient as with Guanidine-HCl. In urea, the solubilized rshgAd sample contains essentially soluble aggregates and very little amount of monodispersed molecules (see FIG. 4). During the refolding step, these soluble aggregates will not be able to refold and precipitate, leading thus to miserable refolding yields.

In another preferred embodiment, the present invention is directed to a method according to the invention, wherein said second solution comprises sodium acetate. Preferably, said second solution comprises about 500 mM, 400 mM, 300 mM, 200 mM, 100 mM or 50 mM sodium acetate (also referred to as Na acetate) Most preferably, said second solution comprises about 100 mM sodium acetate

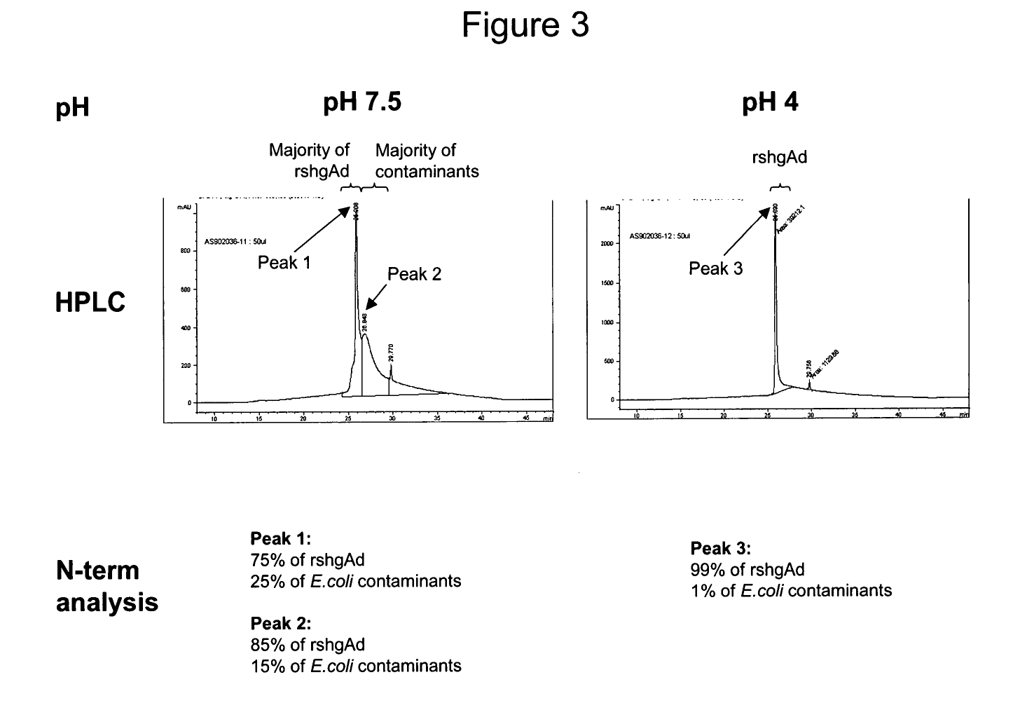

Another preferred embodiment is directed to a method according to the invention, wherein the pH of said second solution is of about 6.5, 6, 5, 4 or 3. Preferably, the pH of said second solution is of about 4. The pH used at the IB solubilization step had a major impact on the final product. Different sets of contaminant proteins were segregated as a function of the pH. Solubilization of the IBs at pH 7.5 had a major negative effect on the final product, which contained up to 10% of HSP-A (E. coli Heat Shock Protein-A) and 15% E. coli 30S ribosomal protein S6 (see FIG. 3). On the other hand when the solubilization was performed at pH 4.0, the percentage of HSP-A in the final product was lower than 1% (see FIG. 3).

In a most preferred embodiment, the second solution is a solution at pH 4 comprising 100 mM Na Acetate, 1 mM DTT and 6 M Guanidine-HCl.

### 2.3. Desalting Step

A preferred embodiment is directed to a method according to the invention, wherein said third solution comprises about 8 M, 7 M, 6 M urea. Preferably, said third solution comprises about 8 M urea.

In a preferred embodiment, the present invention is directed to a method according to the invention, wherein the pH of said third solution is of about 5, 4 or 3. Preferably, the pH of said third solution is of about 4.

In another preferred embodiment, the present invention is directed to a method according to the invention, wherein said third solution exhibits a low ionic strength. As used herein, the term “low ionic strength” refers to a solution wherein the buffer concentration (e.g., acetic acid concentration) has a value equal or inferior to 50 mM.

In another preferred embodiment, the present invention is directed to a method according to the invention, wherein said third solution comprises acetic acid. Preferably, said third solution comprises about 50 mM, 40 mM, 30 mM, 20 mM, 10 mM, 5 mM acetic acid. Most preferably, said third solution comprises about 20 mM acetic acid.

In a most preferred embodiment, the third solution is a solution at pH 4 comprising 20 mM acetic acid, 5 mM DTT and 8 M urea.

In a most preferred embodiment, step f) is carried out by passaging the solubilized IBs through a size exclusion chromatography column equilibrated with the third solution. When step f) is carried out by passaging the solubilized IBs through a size exclusion chromatography column equilibrated with the third solution, the fractions comprising gAdiponectin polypeptides obtained at the end of the SEC are pooled. Preferably, these fractions comprise a majority of monodispersed gAdiponectin polypeptides as compared to aggregates (see FIG. 4). This pooling step is optional in case only one fraction comprises gAdiponectin polypeptides.

2.4. Refolding of the gAdiponectin Polypeptides

A preferred embodiment is directed to a method according to the invention wherein droplets of the solution obtained at the end of step (f) is added to a bath of said fourth solution.

In a preferred embodiment, 1 volume of said solution obtained at the end of step (f) is added into 3, 4, 6, 8, 10, 12, 14 or 16 volumes of said fourth solution. The solution obtained at the end of step (f) is added progressively into the fourth solution over a period of time of about 24, 20, 18, 16, 14, 12, 10, 8, 6 or four hours. Preferably, the solution obtained at the end of step (f) is added progressively into the fourth solution over a period of time of about 12 hours.

In another preferred embodiment, the present invention is directed to a method according to the invention wherein the final concentration of said gAdiponectin polypeptides in said fourth solution is about 150, 125, 100, 75, 50 or 25 μg/ml. Preferably, the final concentration of said gAdiponectin polypeptides in said fourth solution is about 100 μg/ml.

In still another preferred embodiment, the present invention is directed to a method according to the invention wherein step (g) is carried out at about 25, 20, 15, 10, 5 or 4° C. Preferably, step (g) is carried out at about 5° C. or at about 4° C.

Another preferred embodiment is directed to a method according to the invention wherein the fourth solution comprises glycerol. Preferably, said fourth solution comprises about 15%, 10% or 5% glycerol. Most preferably, said fourth solution comprises about 10% glycerol.

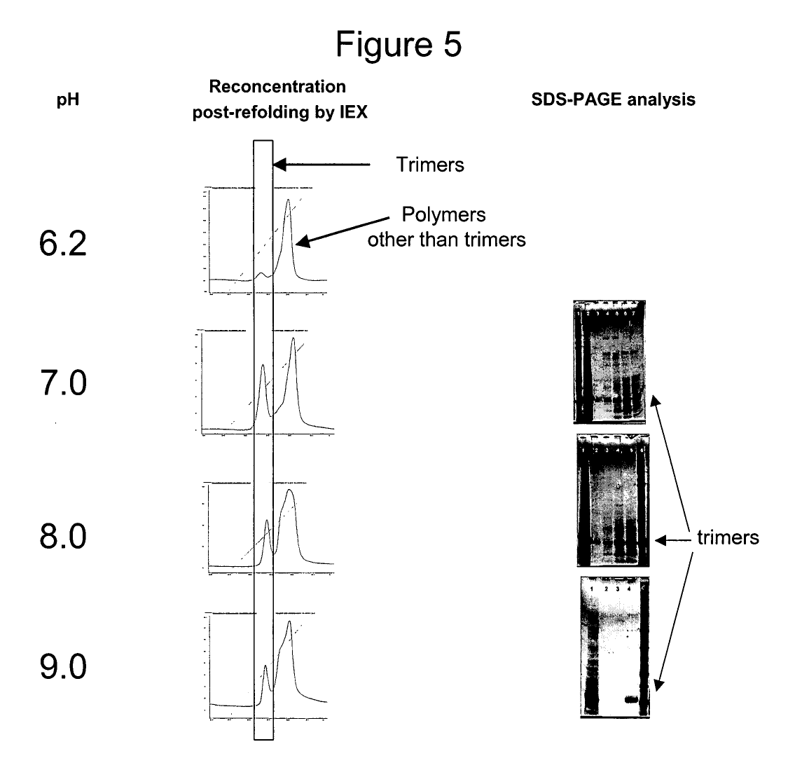

Still another preferred embodiment is directed to a method according to the invention wherein the pH of said fourth solution is of about 10, 9 or 8. Preferably, the pH of said fourth solution is of about 9. As shown on FIG. 5, a pH of 9 allows obtaining a fraction comprising pure trimers.

In a most preferred embodiment, the fourth solution is a solution at pH 9 comprising 100 mM ethanolamine, 1 mM DTT and 10% glycerol.

### 2.5. Chromatography Columns

In a preferred embodiment, a Size Exclusion Chromatography (SEC) is carried out at step (f) of the method according to the invention. The SEC is preferably performed on a Sephadex-G25 gel filtration column (Amersham Biosciences; Reference No. 17-0033) or the like. The second solution is replaced by the third solution by desalting on said Sephadex-G25 gel filtration column, on which the sample volume corresponds to 4% of the column volume.

In another preferred embodiment, the Anion Exchange Chromatography (AEC) carried out at step (h) of the method according to the invention is performed using a weak ion exchange chromatography (IEX) resin. Preferably, said AEC is performed on a Fractogel EMD DEAE column (Merck; Reference No. 1.16883) or the like.

In still another preferred embodiment, the SEC carried out at step (j) of the method according to the invention is performed on a Superdex 200 prepacked column (Amersham Biosciences Reference Nos. 17-1069-01 and 17-1071-01) or the like. Both improvement of the purity of the gAdiponectin polypeptides and calibration of the final material is obtained by carrying out step (j).

3. Process of the Second Embodiment

This embodiment is illustrated by Example 2. This embodiment is characterized by a supplemental purification step: a SEC performed between steps (e) and (f) of the method in accordance with the invention. The conditions used for steps (a) to (k) may be the same as those of the process of the first embodiment, or may be different as further detailed below.

### 3.1. Wash of the IBs

A preferred embodiment is directed to a method of producing a recombinant gAdiponectin polypeptide according to the invention wherein the pH of said first solution is of about 8, 7.5 or 7. Preferably, the pH of said first solution is of about 7.5.

In a most preferred embodiment, the first solution is a solution at pH 7.5 comprising 100 mM Tris/HCl, 1 M urea, 5 mM DTT and 1 mM NaN₃.

### 3.2. Solubilization of the IBs

In a preferred embodiment, the second solution is identical to any of the solutions described in paragraph 2.2. hereabove.

### 3.3. Supplemental SEC

A preferred embodiment is directed to a method in accordance with the invention wherein the solubilized IBs obtained at the end of step (e) are passed through a size exclusion chromatography column before carrying out step (f), wherein said size exclusion chromatography column is equilibrated with a sixth solution comprising Guanidine-HCl.

Preferably, the supplemental SEC is equilibrated with a solution that is identical to any of the second solutions described in paragraph 2.2. hereabove.

Also preferably, said supplemental SEC is performed using a Sephacryl S-200HR column or the like.

### 3.4. Desalting Step

A preferred embodiment is directed to a method according to the invention, wherein said third solution comprises about 8 M, 7 M, 6 M urea. Preferably, said third solution comprises about 8 M urea.

In another preferred embodiment, the present invention is directed to a method according to the invention, wherein the pH of said third solution is of about 5, 4 or 3. Preferably, the pH of said third solution is of about 4.

In another preferred embodiment, the present invention is directed to a method according to the invention, wherein said third solution exhibits a low ionic strength.

In another preferred embodiment, the present invention is directed to a method according to the invention, wherein said third solution comprises acetic acid. Preferably, said third solution comprises about 50 mM, 40 mM, 30 mM, 20 mM, 10 mM, 5 mM acetic acid. Most preferably, said third solution comprises about 50 mM acetic acid.

In a most preferred embodiment, the third solution is a solution at pH 4 comprising 50 mM acetic acid, 5 mM DTT and 8 M urea. Alternatively, the third solution may be a solution at pH 4 comprising 20 mM acetic acid, 5 mM DTT and 8 M urea.

Most preferably, step (f) is carried out by passaging the solubilized IBs through a size exclusion chromatography column equilibrated with the third solution.

3.5. Refolding of the gAdiponectin Polypeptides

A preferred embodiment is directed to a method according to the invention wherein droplets of solution obtained at the end of step (f) is added to a bath of said fourth solution.

In a preferred embodiment, 1 volume of said solution obtained at the end of step (f) is added into 4, 6, 8, 10, 12, 14 or 16 volumes of said fourth solution. The solution obtained at the end of step (f) is added progressively into the fourth solution over a period of time of about 24, 20, 18, 16, 14, 12, 10, 8, 6 or 4 hours. Preferably, the solution obtained at the end of step (f) is added progressively into the fourth solution over a period of time of about 12 hours.

In another preferred embodiment, the present invention is directed to a method according to the invention wherein the final concentration of said gAdiponectin polypeptides in said fourth solution is about 75, 50, 25, 20 or 10 μg/ml. Preferably, the final concentration of said gAdiponectin polypeptides in said fourth solution is about 25 μg/ml.

In still another preferred embodiment, the present invention is directed to a method according to the invention wherein step (g) is carried out at about 6, 5, 4, 3, 2, 1 or 0° C. Preferably, step (g) is carried out at about 5° C. or at about 4° C.

Still another preferred embodiment is directed to a method according to the invention wherein the pH of said fourth solution is of about 10, 9, 8 or 7.5. Preferably, the pH of said fourth solution is of about 9.

In a most preferred embodiment, the fourth solution is a solution at pH 9 comprising 100 mM ethanolamine and 1 mM DTT.

Optionally, the fourth solution comprises glycerol. Preferably, said fourth solution comprises about 25%, 20%, 15%, 10% or 5% glycerol. Most preferably, said fourth solution comprises about 10% glycerol.

### 3.6. Chromatography Columns

In a preferred embodiment, the chromatography columns are identical to the columns described in paragraph 2.5. hereabove.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### E. coli Strain

An E. coli strain was transformed with an expression vector comprising a sequence encoding a polypeptide of SEQ ID NO: 2. The protein was expressed at small scale, purified and its N-terminal extremity sequenced. The N-terminal extremity of the expressed proteins was homogenous. There was a complete removal of the N-term Methionine. Thus the produced protein, referred to as rshgAd, consist of amino acids 2 to 138 of SEQ ID NO: 2.

### Example 2

### Process of the Second Embodiment

2.1. Cell Culture

The above E. coli strain was cultivated in a 50 L bioreactor. After centrifugation, a cell pellet of 1871 g was obtained. An aliquot of 227 g was used in the process described below.

2.2. Purification Process

The thawed cell paste was resuspended at a ratio of 1 g of thawed cell paste for 5.6 ml of 100 mM Tris/HCl, pH 7.5, 5.0 mM DTT, 1.0 mM Na N3 (Buffer A). The suspension was homogenized in order to obtain a suspension devoid of clumps. All these manipulations were carried out at 4° C. in the presence of Benzonase® nuclease. The bacterial suspension was lysed by passing it three times through an APV Lab 2000 mechanical disrupter (APV Invensys, Worb, Switzerland) at 1600 bars that had been pre-cooled at 4° C.

The cell lysate was then centrifuged at 27'500×g for 60 min. The pellets comprise the inclusion bodies (IBs) containing the rshgAd proteins.

The pellets were washed six times with 100 mM Tris/HCl, pH 7.5 containing with 1 M urea, 5 mM DTT and 1.0 mM NaN₃. The final cell concentration was of 1 g of cell paste in 25 to 30 ml of buffer.

The IBs recovered in the final washed pellets were solubilized at a concentration of 1 g of IBs in 31.0 ml of 100 mM Na Acetate pH 4.0, 1 mM DTT, 6 M Guanidine-HCl (Buffer B). The suspension was heated to 60° C. with stirring for 1 hr, and incubated overnight at room temperature.

After centrifugation at 100'000×g for 60 min, the solubilized IB were recovered in the supernatant which were purified by Size Exclusion Chromatography (SEC) in several runs of 90 ml on a Sephacryl S-200HR column (5 cm diameter×90 cm=1'800 ml) equilibrated in Buffer B (GE-Healthcare Reference No. 17-0584-01, instructions 52-2086-00 AK).

The fractions containing the monodisperse rshgAd proteins were pooled and desalted into 50 mM Acetic acid, 5 mM DTT, 8 M urea, pH 4.0 (Buffer C) onto a XK50/30 column packed with the Sephadex® G-25 medium resin (Amersham Biosciences; Reference No. 17-0033) equilibrated and run in Buffer C.

The fractions containing rshgAd, whose purity was higher than 95% were pooled. The pool was quantified by Coomassie blue protein assay to determine the total protein. RshgAd content was measured using SDS-PAGE including rshgAd calibration curve.

RshgAd refolding started by adjustment of the protein concentration to 400 μg/ml with buffer C. For a final renaturation concentration of 25 μg/ml, the protein solution (volume=X) is applied dropwise into a refolding bath filled with 16× volume of buffer D (50 mM Ethanolamine, 1 mM DTT, pH 9.0) overnight, at 0.8 ml/min at 4° C.

The next day, the refolding solution was filtered through a 0.22 μm filter (Filtration on SpiralCap PF Capsule 0.8-0.2 μm Pall) to remove mis-folded and aggregated proteins.

The soluble refolding solution was concentrated by anion exchange chromatography using Q-Sepharose run (GE-Healthcare Reference No. 17-0510-01; instructions 71-7070-00) in buffer D (50 mM Ethanolamine, 1 mM DTT, pH 9.0). The protein was eluted with a 0-1 M salt gradient, and rshgAd eluted at 0.3 M NaCl.

Finally, the rshgAd refolding solution was concentrated on YM10 ultrafiltration membrane (Millipore) and applied to a Superdex 200 prepacked column (5 cm diam.×90 cm; Amersham Biosciences Reference Nos. 17-1069-01 and 17-1071-01) equilibrated in 100 mM Tris, 1 mM DTT, pH 8.5. Trimeric rshgAd (MW˜40'000) was pooled, reconcentrated on YM 10 ultrafiltration membrane to 1.0 mg/ml, aliquoted and stored at −80° C.

2.3. Results

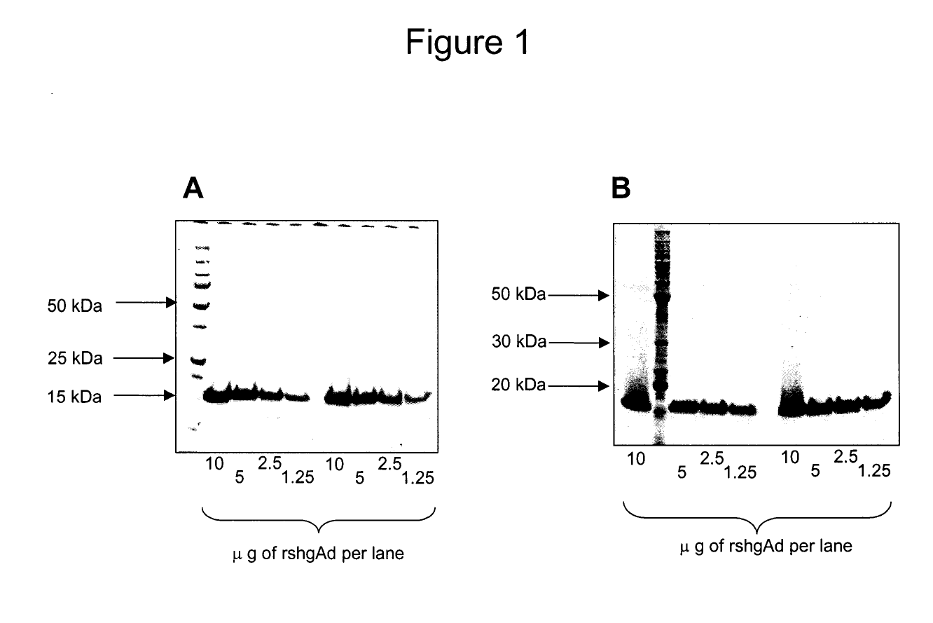

The obtained protein was analyzed by SDS-PAGE according to the manufacturer's instructions (NuPAGE®Bis-Tris Gels and NuPAGE®Buffers; References No. NP0301 Gels and NP0002). The results are shown in FIG. 1A.

As shown in Table 1 below, the process allowed obtaining 359 mg of rshgAd protein. When the purity was measured by densitometric scanning of the SDS-PAGE gel according to the manufacturer's instructions (BIO-RAD densitometer GS-800, Reference No. 170-7980, Manual 4000188), a purity of 100% was measured.

TABLE 1

| | | | | | rhgAd | | | | |
|---|---|---|---|---|---|---|---|---|---|
| | Weight | | Total protein | | content | | | | |
| | (g | Volume | (mg/ml) | | (mg/ml) | | | Refolding | Purification |
| Purification step | w/w) | (ml) | (mgs) | | (mgs) | | Purity % | yield % | yield % |
| Cell paste | 227.4 | 1280 | 18.20 | 18.20 | 14.65 | 18748 | 57.0 | — | 100.0 |
| Washed pellet | 90.3 | 1530 | 7.67 | 7.67 | 5.35 | 8179 | 68.3 | — | 43.6 |
| resuspended in | | | | | | | | | |
| 6M Guanidine | | | | | | | | | |
| Solubilized IB′s | 61.8 | 1420 | 6.57 | 6.57 | 5.11 | 7252 | 80.5 | — | 38.7 |
| in 6M | | | | | | | | | |
| Guanidine-HCl | | | | | | | | | |
| SEC S200HR in | — | 6904 | 0.61 | 4239 | 0.71 | 4902 | 90.0 | — | 26.1 |
| 6M Guanidine- | | | | | | | | | |
| HCl | | | | | | | | | |
| Desalting | — | 11835 | 0.36 | 4201 | 0.41 | 4852 | 95.0 | — | 25.9 |
| Guanidine-HCl | | | | | | | | | |
| to Urea | | | | | | | | | |
| Refolding | — | 168057 | 0.03 | 4201 | 0.03 | 4201 | 95.0 | 100.0 | 22.4 |
| Anion | — | 4322 | 0.20 | 847 | 0.20 | 847 | 95.0 | 16.6 | 4.5 |
| exchanger- | | | | | | | | | |
| Reconcentration | | | | | | | | | |
| SEC on | — | 1662 | 0.22 | 359 | 0.22 | 359 | 100.0 | 7.7 | 1.9 |
| Superdex-200 | | | | | | | | | |
| (rhgAd trimers) | | | | | | | | | |

Table 2 below shows that theoretically, about 14 g of trimers of rhgAd could be obtained from 100 L of cell culture.

TABLE 2

| | Final amount of | Purification | Purification | |
|---|---|---|---|---|
| | refolded trimeric | yield | yield | Purification yield |
| Initial amount of | rhsAd | (mg/g cell | (mg/L | (g trimers rhgAd/100 L |
| cell paste (g) | (mgs) | paste) | fermentation) | fermentation) |
| 227.4 | 359 | 1.6 | 142.0 | 14.2 |

The biological activity of the rhgAd polypeptide was tested using the epinephrine-induced hyperglycemia model in mice (Kuhn et al., 1987). Acute stress hyperglycemia was mimicked by the injection of epinephrine (0.2 mg/kg, subcutaneous route) to 4-hour fasted C57BL/6 mice (9-11 week old). Thirty minutes later blood was sampled under isoflurane anesthesia and glucose level determined using a glucometer. The rhgAd polypeptides were administered by subcutaneous route.

As shown in Table 3, the rhgAd polypeptides were found to be biologically active. A rhgAd dose of 0.1 mg·kg⁻¹ allowed obtaining an inhibition of 54% of the epinephrine-induced hyperglycemia. The inhibition was dose-dependent.

TABLE 3

| | | | | Blood Glucose 30 mn | | |
|---|---|---|---|---|---|---|
| | Dose | Animal | Body | Vehicle | Product | Inhibition |
| Product | Mg/ml | number | weight g | mM | mM | % |
| Baseline | 0 | 4 | 23.4 | 8.0 | 8.0 | 100 |
| vehicle | | | 0.3 | 1.2 | 1.2 | |
| Control | 0 | 7 | 23.9 | 8.0 | 14.6 | 0 |
| vehicle | | | 0.5 | 1.2 | 1.2 | |
| rhgAd | 0.01 | 7 | 23.2 | 8.0 | 14.2 | 6 |
| | | | 0.5 | 1.2 | 1.2 | |
| rhgAd | 0.03 | 7 | 23.0 | 8.0 | 11.5 | 47 |
| | | | 0.2 | 1.2 | 1.3 | |
| rhgAd | 0.1 | 7 | 23.4 | 8.0 | 11.0 | 54 |
| | | | 0.6 | 1.2 | 1.0 | |

In conclusion, this process allows obtaining high yields of pure gAdiponectin polypeptides that are biologically active.

### Example 3

### Process of the First Embodiment

3.1. Cell Culture

The E. coli strain of Example 1 was cultivated in a 50 L bioreactor. After centrifugation, a cell pellet of 1871 g was obtained. An aliquot of 321 g was used in the process described below.

3.2. Purification Process

The thawed cell paste was resuspended at a ratio of 1 g of thawed cell paste for 5.6 ml of 100 mM Tris/HCl, pH 7.5, 5.0 mM DTT, 1.0 mM Na N3 (Buffer A). The suspension was homogenized with a Polytron to obtain a slurry devoid of fragments/clumps. All manipulations were carried out at 4° C. in the presence of Benzonase (48 U/g cell paste).

The bacterial suspension was then lysed with three passages through an APV Lab 2000 mechanical disrupter at 1600 bars pre-cooled at 4° C. The cell lysate was centrifuged at 27'500×g for 60 min.

The pellets were washed four times. The final cell paste concentration was of 1 g of cell paste in 14-16 ml of 100 mM Tris/HCl at pH 7.5 containing 20% Ethanol. The inclusion bodies (IBs) recovered in the final washed pellets were solubilized at a concentration of 1 g of IBs in 15.5 ml of 100 mM Na Acetate pH 4.0, 10 mM DTT, 6 M Guanidin-HCl (Buffer B). The suspension was heated to 60° C. with stirring for 1 hr, and further incubated overnight at room temperature. After centrifugation at 100'000×g for 60 min, the solubilized IB were recovered in the supernatant and filtered through a 0.22 μm filter.

The soluble extract was desalted into 20 mM Acetic acid, 5 mM DTT, 8 M urea, pH 4.0 (Buffer C) onto a Sephadex G-25 medium resin packed in an XK50/30 column (1 column volume=490 ml resin) equilibrated and run in Buffer C. The central part of the peak is collected with the aim to have material at the highest possible concentration, ideally 0.8 mg/ml.

The fractions containing rshgAd the purity of which was higher than 87% were pooled. The concentration of the pool was determined both by a Coomassie blue protein assay for total protein estimation followed by an SDS-PAGE analysis under reducing conditions. The SDS-PAGE was stained by Coomassie blue and included a calibration curve of rshgAd standards in the gel. Protein and urea concentrations of the pool were adjusted. First the protein concentration was adjusted to 800 μg/ml with Buffer C. Then both the urea and the protein concentration are adjusted to 4M and 400 μg/ml respectively using 20 mM Acetic acid, 5 mM DTT, pH 4.0 (Buffer C′). This buffer was slowly added to the protein solution.

Refolding was done by infinite dilution of the protein solution, which was added dropwise into a refolding bath containing 100 mM Ethanolamine, 1 mM DTT, 10% glycerol, pH 9.0 (buffer D). For a final refolding concentration of 100 μg/ml, the protein solution (volume=1×) was added overnight at 0.8 ml/min at 4° C. to the bath filled with 4× volume of buffer D. The next day, the refolding solution was filtered through a 0.22 mm filter (Filtration on SpiralCap PF Capsule 0.8-0.2 μm Pall) to remove miss-folded and aggregated proteins.

The soluble refolding solution was concentrated by anion exchange chromatography using Fractogel EMD DEAE (Merck; Reference No. 1.16883) run in buffer E (100 mM Tris-HCl, 1 mM DTT, pH 8.5). The protein was eluted with a 0-1 M salt gradient, where rshgAd eluted at 0.2 M NaCl.

Finally, the rshgAd solution was concentrated on YM10 ultrafiltration membrane (Millipore) and applied to a Superdex 200 column (26 mm diam.×90 cm) equilibrated in 100 mM Tris-HCl, pH 8.5. Trimeric rshgAd (MW˜40'000) was pooled, reconcentrated on YM 10 ultrafiltration membrane to 1.0 mg/ml, aliquoted and stored at −80° C.

3.3. Results

The obtained protein was analyzed by SDS-PAGE. The results are shown in FIG. 1B.

As shown in Table 4 below, the process allowed obtaining more than 1 g of rshgAd protein. When the purity was measured by densitometric scanning of the SDS-PAGE gel, a purity of 98% was measured.

TABLE 4

| | | | | | rhgAd | | | | |
|---|---|---|---|---|---|---|---|---|---|
| | Weight | | Total protein | | content | | | | |
| | (g | Volume | (mg/ml) | | (mg/ml) | | | Refolding | Purification |
| Purification step | w/w) | (ml) | (mgs) | | (mgs) | | Purity % | yield % | yield % |
| Cell paste | 321.2 | 1300 | 24.69 | 32091 | 15.10 | 19625 | 48.7 | — | 100.0 |
| Washed pellet | 136.5 | 2115 | 9.01 | 19048 | 5.51 | 11643 | 60.3 | — | 59.3 |
| resuspended in | | | | | | | | | |
| 6M Guanidine | | | | | | | | | |
| Solubilized IB′s | — | 1962 | 8.38 | 16442 | 5.12 | 10045 | 85.0 | — | 51.2 |
| in 6M | | | | | | | | | |
| Guanidine-HCl | | | | | | | | | |
| Desalting | — | 5038 | 1.43 | 7220 | 1.25 | 6298 | 87.2 | — | 32.1 |
| Guanidine-HCl | | | | | | | | | |
| to Urea | | | | | | | | | |
| Refolding | — | 62978 | 0.14 | 9025 | 0.10 | 6298 | 87.2 | 100.0 | 32.1 |
| Anion | — | 9840 | 0.20 | 1934 | 0.18 | 1732 | 89.5 | 27.5 | 8.8 |
| exchanger- | | | | | | | | | |
| Reconcentration | | | | | | | | | |
| SEC on | — | 2755 | 0.41 | 1119 | 0.41 | 1119 | 98.0 | 16.9 | 5.7 |
| Superdex-200 | | | | | | | | | |
| (rhgAd trimers) | | | | | | | | | |

Table 5 below shows that theoretically, nearly 30 g of trimers of rhgAd could be obtained from 100 L of cell culture.

TABLE 5

| | Final amount of | Purification | Purification | |
|---|---|---|---|---|
| | refolded trimeric | yield | yield | Purification yield |
| Initial amount of | rhsAd | (mg/g cell | (mg/L | (g trimers rhgAd/100 L |
| cell paste (g) | (mgs) | paste) | fermentation) | fermentation) |
| 321.2 | 1065 | 3.3 | 298.1 | 29.8 |

The biological activity of the rhgAd polypeptide was tested using the epinephrine-induced hyperglycemia model in mice, and the rhgAd polypeptides were found to be biologically active (see Table 6). A rhgAd dose of 0.1 mg·kg⁻¹ allowed obtaining an inhibition of 89% of the epinephrine-induced hyperglycemia. The inhibition was dose-dependent.

TABLE 6

| | | | | Blood Glucose 30 mn | | |
|---|---|---|---|---|---|---|
| | Dose | Animal | Body | Vehicle | Product | Inhibition |
| Product | Mg/ml | number | weight g | mM | mM | % |
| Baseline | 0 | 4 | 18.1 | 7.6 | 7.6 | 100 |
| vehicle | | | 0.3 | 0.6 | 0.6 | |
| Control | 0 | 8 | 18.0 | 7.6 | 15.2 | 0 |
| vehicle | | | 0.3 | 0.6 | 1.0 | |
| rhgAd | 0.01 | 7 | 18.1 | 7.6 | 11.8 | 43 |
| | | | 0.1 | 0.6 | 1.0 | |
| rhgAd | 0.03 | 7 | 18.1 | 7.6 | 10.3 | 61 |
| | | | 0.2 | 0.6 | 1.3 | |
| rhgAd | 0.1 | 7 | 18.1 | 7.6 | 7.2 | 89 |
| | | | 0.1 | 0.6 | 0.6 | |

This process thus allows obtaining high yields of pure gAdiponectin polypeptides that are biologically active.

### REFERENCES

Altschul, S. F., Gish, W., Miller, W., Myers, E. W., and Lipman, D. J. (1990). Basic local alignment search tool. J. Mol. Biol. 215, 403-410.
Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
Arita, Y., Kihara, S., Ouchi, N., Takahashi, M., Maeda, K., Miyagawa, J., Hotta, K., Shimomura, I., Nakamura, T., Miyaoka, K., Kuriyama, H., Nishida, M., Yamashita, S., Okubo, K., Matsubara, K., Muraguchi, M., Ohmoto, Y., Funahashi, T., and Matsuzawa, Y. (1999). Paradoxical decrease of an adipose-specific protein, adiponectin, in obesity. Biochem. Biophys. Res. Commun. 257, 79-83.
Berg, A. H., Combs, T. P., Du, X., Brownlee, M., and Scherer, P. E. (2001). The adipocyte-secreted protein Acrp30 enhances hepatic insulin action. Nat. Med. 7, 947-953.
Brutlag, D. L., Dautricourt, J. P., Maulik, S., and Relph, J. (1990). Improved sensitivity of biological sequence database searches. Comput. Appl. Biosci. 6, 237-245.
Fruebis, J., Tsao, T. S., Javorschi, S., Ebbets-Reed, D., Erickson, M. R., Yen, F. T., Bihain, B. E., and Lodish, H. F. (2001). Proteolytic cleavage product of 30-kDa adipocyte complement-related protein increases fatty acid oxidation in muscle and causes weight loss in mice. Proc. Natl. Acad. Sci. U.S.A 98, 2005-2010.
Gonnet, G. H., Cohen, M. A., and Benner, S. A. (1992). Exhaustive matching of the entire protein sequence database. Science 256, 1443-1445.
Grantham, R. (1974). Amino acid difference formula to help explain protein evolution. Science 185, 862-864.
Hara, K., Boutin, P., Mori, Y., Tobe, K., Dina, C., Yasuda, K., Yamauchi, T., Otabe, S., Okada, T., Eto, K., Kadowaki, H., Hagura, R., Akanuma, Y., Yazaki, Y., Nagai, R., Taniyama, M., Matsubara, K., Yoda, M., Nakano, Y., Tomita, M., Kimura, S., Ito, C., Froguel, P., and Kadowaki, T. (2002). Genetic variation in the gene encoding adiponectin is associated with an increased risk of type 2 diabetes in the Japanese population. Diabetes 51, 536-540.
Henikoff, S. and Henikoff, J. G. (1993). Performance evaluation of amino acid substitution matrices. Proteins 17, 49-61.
Higgins, D. G., Thompson, J. D., and Gibson, T. J. (1996). Using CLUSTAL for multiple sequence alignments. Methods Enzymol. 266, 383-402.
Hu, E., Liang, P., and Spiegelman, B. M. (1996). AdipoQ is a novel adipose-specific gene dysregulated in obesity. J. Biol. Chem. 271, 10697-10703.
Karlin, S. and Altschul, S. F. (1990). Methods for assessing the statistical significance of molecular sequence features by using general scoring schemes. Proc. Natl. Acad. Sci. U.S.A 87, 2264-2268.
Kondo, H., Shimomura, I., Matsukawa, Y., Kumada, M., Takahashi, M., Matsuda, M., Ouchi, N., Kihara, S., Kawamoto, T., Sumitsuji, S., Funahashi, T., and Matsuzawa, Y. (2002). Association of adiponectin mutation with type 2 diabetes: a candidate gene for the insulin resistance syndrome. Diabetes 51, 2325-2328.
Kuhn, C. M., Cochrane, C., Feinglos, M. N., and Surwit, R. S. (1987). Exaggerated peripheral responses to catecholamines contributes to stress-induced hyperglycemia in the ob/ob mouse. Pharmacol. Biochem. Behav. 26, 491-495.
Lindsay, R. S., Funahashi, T., Hanson, R. L., Matsuzawa, Y., Tanaka, S., Tataranni, P. A., Knowler, W. C., and Krakoff, J. (2002). Adiponectin and development of type 2 diabetes in the Pima Indian population. Lancet 360, 57-58.
Liu, H., Li, X., Song, H., and Tang, Q. (2006). Expression, Purification And Characterization Of Recombinant Human Globular Domain of Adiponectin. Fudan Univ J Med Sci 33, 97-100.
Maeda, K., Okubo, K., Shimomura, I., Funahashi, T., Matsuzawa, Y., and Matsubara, K. (1996). cDNA cloning and expression of a novel adipose specific collagen-like factor, apM1 (AdiPose Most abundant Gene transcript 1). Biochem. Biophys. Res. Commun. 221, 286-289.
Maeda, N., Shimomura, I., Kishida, K., Nishizawa, H., Matsuda, M., Nagaretani, H., Furuyama, N., Kondo, H., Takahashi, M., Arita, Y., Komuro, R., Ouchi, N., Kihara, S., Tochino, Y., Okutomi, K., Horie, M., Takeda, S., Aoyama, T., Funahashi, T., and Matsuzawa, Y. (2002). Diet-induced insulin resistance in mice lacking adiponectin/ACRP30. Nat. Med. 8, 731-737.
Neumeier, M., Weigert, J., Schaffler, A., Wehrwein, G., Muller-Ladner, U., Scholmerich, J., Wrede, C., and Buechler, C. (2006). Different effects of adiponectin isoforms in human monocytic cells. J. Leukoc. Biol. 79, 803-808.
Pajvani, U. B., Du, X., Combs, T. P., Berg, A. H., Rajala, M. W., Schulthess, T., Engel, J., Brownlee, M., and Scherer, P. E. (2003). Structure-function studies of the adipocyte-secreted hormone Acrp30/adiponectin. Implications for metabolic regulation and bioactivity. J. Biol. Chem. 278, 9073-9085.
Panidis, D., Kourtis, A., Farmakiotis, D., Mouslech, T., Rousso, D., and Koliakos, G. (2003). Serum adiponectin levels in women with polycystic ovary syndrome. Hum. Reprod. 18, 1790-1796.
Pearson, W. R. and Lipman, D. J. (1988). Improved tools for biological sequence comparison. Proc. Natl. Acad. Sci. U.S. A 85, 2444-2448.
Petridou, E., Mantzoros, C., Dessypris, N., Koukoulomatis, P., Addy, C., Voulgaris, Z., Chrousos, G., and Trichopoulos, D. (2003). Plasma adiponectin concentrations in relation to endometrial cancer: a case-control study in Greece. J. Clin. Endocrinol. Metab 88, 993-997.
Ramsay, J. E., Jamieson, N., Greer, I. A., and Sattar, N. (2003). Paradoxical elevation in adiponectin concentrations in women with preeclampsia. Hypertension 42, 891-894.
Scherer, P. E., Williams, S., Fogliano, M., Baldini, G., and Lodish, H. F. (1995). A novel serum protein similar to C1q, produced exclusively in adipocytes. J. Biol. Chem. 270, 26746-26749.
Thompson, J. D., Higgins, D. G., and Gibson, T. J. (1994). CLUSTAL W: improving the sensitivity of progressive multiple sequence alignment through sequence weighting, position-specific gap penalties and weight matrix choice. Nucleic Acids Res. 22, 4673-4680.
Xu, A., Wang, Y., Keshaw, H., Xu, L. Y., Lam, K. S., and Cooper, G. J. (2003). The fat-derived hormone adiponectin alleviates alcoholic and nonalcoholic fatty liver diseases in mice. J. Clin. Invest 112, 91-100.
Yamauchi, T., Kamon, J., Waki, H., Terauchi, Y., Kubota, N., Hara, K., Mori, Y., Ide, T., Murakami, K., Tsuboyama-Kasaoka, N., Ezaki, O., Akanuma, Y., Gavrilova, O., Vinson, C., Reitman, M. L., Kagechika, H., Shudo, K., Yoda, M., Nakano, Y., Tobe, K., Nagai, R., Kimura, S., Tomita, M., Froguel, P., and Kadowaki, T. (2001). The fat-derived hormone adiponectin reverses insulin resistance associated with both lipoatrophy and obesity. Nat. Med. 7, 941-946.
Yokota, T., Oritani, K., Takahashi, I., Ishikawa, J., Matsuyama, A., Ouchi, N., Kihara, S., Funahashi, T., Tenner, A. J., Tomiyama, Y., and Matsuzawa, Y. (2000). Adiponectin, a new member of the family of soluble defense collagens, negatively regulates the growth of myelomonocytic progenitors and the functions of macrophages. Blood 96, 1723-1732.
Zoccali, C., Mallamaci, F., Panuccio, V., Tripepi, G., Cutrupi, S., Parlongo, S., Catalano, F., Tanaka, S., Ouchi, N., Kihara, S., Funahashi, T., and Matsuzawa, Y. (2003). Adiponectin is markedly increased in patients with nephrotic syndrome and is related to metabolic risk factors. Kidney Int. Suppl S98-102.

1-26. (canceled)

27. A method of producing a recombinant polypeptide wherein said method comprises the steps of;
a) cultivation of recombinant E. coli cells expressing said recombinant polypeptide;
b) lysis of said cells;
c) recovery of inclusion bodies (IBs) comprising said recombinant polypeptides;
d) washing of said IBs in a first solution;
e) solubilization of said IBs in a second solution;
f) buffer exchange of the solubilized IBs into a third solution;
g) refolding of said recombinant polypeptide by adding the solution obtained at the end of step (f) into a fourth solution;
h) concentration of said recombinant polypeptides by passing the solution obtained at the end of step (g) through an anion exchange chromatography column; and
i) recovery of the fractions comprising said recombinant polypeptides,
wherein said process is characterized in that:
i) said recombinant polypeptide is a polypeptide comprising the globular head of Adiponectin (gAdiponectin);
ii) said second solution comprises guanidine and its pH is acidic;
iii) said third solution comprises urea and its pH is acidic;
iv) said solution obtained at the end of step (f) is added progressively into said fourth solution; and
v) the pH of said fourth solution is basic.

28. The method of claim 27, wherein step (f) comprises the steps of:
i) passage of the solubilized IBs through a size exclusion chromatography column equilibrated with said third solution;
ii) recovery of the fractions comprising said gAdiponectin polypeptides; and
iii) collecting said fractions or pooling of said fractions obtained at step (ii).

29. The method of claim 27, wherein said gAdiponectin polypeptide:
a) comprises amino acids 115 to 244 of SEQ ID NO: 1; and
b) lacks amino acids 1 to 70 of SEQ ID NO: 1.

30. The method of claim 27, wherein said gAdiponectin polypeptide consists of amino acids 2 to 138 of SEQ ID NO: 2.

31. The method of claim 27, wherein said first solution comprises ethanol.

32. The method of claim 31, wherein said first solution comprises about 20% ethanol.

33. The method of claim 27, wherein said first solution is a 100 mM Tris/HCl solution at pH 7.5.

34. The method of claim 27, wherein said second solution comprises about 6 M Guanidine-HCl.

35. The method of claim 27, wherein said second solution comprises sodium acetate.

36. The method of claim 27, wherein the pH of said second solution is of about 4.

37. The method of claim 27, wherein said second solution is a solution at pH 4 comprising 100 mM Na Acetate, 1 mM DTT and 6 M Guanidine-HCl.

38. The method of claim 27, wherein said third solution comprises about 8 M urea.

39. The method of claim 27, wherein the pH of said third solution is of about 4.

40. The method of claim 27, wherein said third solution has a low ionic strength.

41. The method of claim 27, wherein said third solution comprises acetic acid.

42. The method of claim 27, wherein said third solution is a solution at pH 4 comprising 20 mM acetic acid, 5 mM DTT and 8 M urea.

43. The method of claim 27, wherein one (1) volume of said solution obtained at the end of step (f) is progressively added into four (4) volumes of said fourth solution over a period of about 12 hours.

44. The method of claim 27, wherein the final concentration of said gAdiponectin polypeptides in said fourth solution is about 100 μg/ml.

45. The method of claim 27, wherein step (g) is carried out at about 4° C.

46. The method of claim 27, wherein said fourth solution comprises glycerol.

47. The method of claim 27, wherein the pH of said fourth solution is of about 9.

48. The method of claim 27, wherein said fourth solution is a solution at pH 9 comprising 100 mM ethanolamine, 1 mM DTT and 10% glycerol.

49. The method of claim 27, wherein the solution obtained at the end of step (g) is filtered.

50. The method of claim 27, wherein the solution obtained at the end of step (i) is concentrated and/or filtered.

51. The method of claim 27, further comprising passage of the fractions obtained at the end of step (i) through a size exclusion chromatography column recovery of fractions containing said recombinant polypeptides.

52. The method of claim 51, wherein the fractions containing said recombinant polypeptides is concentrated and/or filtered.

53. The method of claim 51, further comprising the formulation of said gAdiponectin polypeptide into a pharmaceutical composition.

54. The method of claim 52, further comprising the formulation of said gAdiponectin polypeptide into a pharmaceutical composition.


SEQUENCE LISTING (3 sequences)

SEQ ID NO: 1 - protein, 244 aa, Homo sapiens
  SIGNAL (1)..(18)
  DOMAIN (42)..(107): Collagen-like
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 138 aa, Artificial
  Artificial polypeptide comprising the globular head of Acrp30
MAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 13 aa, Artificial
  linker sequence
EFGAGLVLGGQFM